(19) United States
(12) Patent Application Publication
(10) Pub. No.: US 2012/0220502 A1
(43) Pub. Date: Aug. 30, 2012

(54) COMPOSITIONS COMPRISING ALKYLALKOXYSULFONATES FOR THE PRODUCTION OF HIGH TEMPERATURE STABLE FOAMS

(75) Inventors: Gabriela Alvarez Jürgenson, Mannheim (DE); Günter Oetter, Frankenthal (DE); Vandana Kurkal-Siebert, Heidelberg (DE); Marta Reinoso Garcia, Dossenheim (DE); Stefan Stein, Worrstadt (DE); Geza Horvath Szabo, Edmonton (CA)

(73) Assignee: BASF SE, Ludwigshafen (DE)

(21) Appl. No.: 13/404,677

(22) Filed: Feb. 24, 2012

Related U.S. Application Data
(60) Provisional application No. 61/446,047, filed on Feb. 24, 2011.

(51) Int. Cl. C09K 8/06; C09K 8/58; C11D 3/60; C07C 309/10; C11D 1/12

(52) U.S. Cl. 507/135; 562/110; 510/495; 510/497; 507/255; 75/330

(57) ABSTRACT

The present invention is directed to the use of alkylalkoxysulfonates for the production of foams, which are stable at high temperatures, e.g. up to about 250° C. Furthermore, the invention relates to a method for producing of high temperature stable foams by using a foamable aqueous composition comprising at least one linear alkylalkoxysulfonate.

### CROSS-REFERENCE TO RELATED APPLICATIONS

This application claims the benefit of U.S. Provisional Application 61/446,047, filed Feb. 24, 2011, which is incorporated by reference.

### BACKGROUND OF THE INVENTION

The present invention is directed to the use of alkylalkoxysulfonates for the production of foams, which are stable at high temperatures, e.g. up to about 250° C. Furthermore, the invention relates to a method for producing of high temperature stable foams by using a foamable aqueous composition comprising at least one linear alkylalkoxysulfonate.

Surfactants are commonly used in cosmetics, pharmaceuticals and detergents, wherein the application temperature normally is below 100° C. Known surfactants for washing, cleaning and foaming applications at temperatures from about 10° to 100° C. usually have at least one C₈₋₁₆ alkyl chain as hydrophobic part. Surfactants with longer alkyl chains are under these conditions often less useful and less interfacial active for such purposes.

Some surfactants for application temperatures above 100° C. have at least one alkyl chain of at least C₁₈, in order to increase the residence time at the interface. Typical applications for high temperature stable surfactants, which are in particular stable at temperatures from 150 to 250° C., are high temperature emulsification of e.g. polymer melts, hot water washing, oil recovery applications and drilling additives.

One important application of a surfactant is the use as foaming agent (also referred to as foaming surfactant), which may facilitate the formation of a foam. Normally a foam is a composition comprising a gas and a liquid, that is formed by trapping gas bubbles in a liquid (e.g. water), wherein the gas bubbles are separated by connected liquid films, the so called lamella. Lamellae of foam comprising a liquid, e.g. water, are normally stabilized by at least one surfactant, which shows film-forming properties. In some cases, the foam can be referred to as a colloidal suspension of a gas in a liquid.

Commonly known foaming agents or foaming surfactants facilitate the formation of foam, when dissolved, particularly in small amounts, in the liquid phase and may enhance the colloidal stability of the foam by inhibition of the coalescences of bubbles. It can be assumed that the foaming surfactant adsorbs in a monolayer at the interface and aggregates into stable foam lamellae. Increased interfacial viscosity provides a mechanical resistance to film thinning and rupturing. Commonly known foaming surfactants for temperatures below 100° C. are for example sodium dodecyl sulfate (SDS), ammonium lauryl sulfate (ALS), sodium lauryl ether sulfate (SLES) and betaines.

Foaming agents, and in particular foaming agents which are able to stabilise foams at high temperature and high pressure, can be used in a variety of applications. Important application sectors for high temperature stable foams are oilfield applications, such as acid stimulation, drilling of subterranean geothermal reservoirs and particularly tertiary oil recovery technologies, such as thermally based oil recovery techniques (e.g. SAGD, “steam assisted gravity drainage”; CSS, “cyclic steam stimulation”), which require performance increase at high temperature conditions. Foam compositions which exhibit a high stability at elevated temperatures, such as above 150° C., are also used in specific applications, such as metal working (e.g. stamping, drawing, forming, bending, rolling, cutting, grinding, punching, sawing, hobbing, reaming, spinning, extruding trepanning, coining, swaging), drilling of subterranean geothermal reservoirs, specific cleaning applications, and fire retardant foams.

A variety of surfactants and surfactants mixtures have been proposed for being used in oilfield applications, especially in oilfield applications using foams at high temperatures.

U.S. Pat. No. 4,201,678 describes a mixture of an amphoteric betaine, a salt of a linear aliphatic or alkyl aryl hydrocarbon sulfonate and, optionally, non-neutralized ammonia. The mixture can be used as foaming agent in foam drilling and work over in high temperature wells with temperatures above 200° C.

WO 1994/18431 is directed to foaming compositions comprising a C₁₀₋₁₆-olefin sulfonate surfactant and a solubilising compound to improve brine tolerance for use in enhanced oil recovery at temperatures from 38° C. to 120° C.

CN-A 1 927 993 describes high-temperature stratum self-foaming compositions and their application in viscous oil exploitation. The compositions comprise alpha-olefin sulfonates or di-alkyl di-phenylether-di-sulfonates as foaming agent.

The document U.S. Pat. No. 5,193,618 discloses a method for recovering hydrocarbons from a reservoir using a steam assisted enhanced oil recovery (EOR) technique, wherein a composition comprises a foam diversion surfactant, in particular an alkyl aromatic sulfonate, and a precipitation-control additive comprising an α-olefin sulfonate dimer.

US 2005/0137114 describes a foam composition in a temperature range of about 80 to 160 F (27° C. to 70° C.) comprising at least one anionic surfactant, at least one cationic surfactant, and one or more zwitterionic compounds, wherein the anionic surfactants were selected from sodium or ammonium alcohol ether sulfates, alkylether sulfonates, alkylaryl sulfonates, and mixtures thereof.

Alkylalkoxysulfonates and methods of their production are known in the prior art. The document DE-A 36 22 439 describes the use of C₈₋₁₈-alkylether sulfonates for cosmetic applications. Document U.S. Pat. No. 4,088,189 describes alkylpolyalkoxyalkyl sulfonates and alkylarylpolyalkoxyalkyl sulfonates for use in a surfactant assisted oil recovery process.

However, there is a high need for superior foaming surfactants and foam compositions, which have a high thermal stability, which can easily be prepared and which can be used for the formation of foams at temperatures from 100° C. up to 250° C. There is in particular a need for uses above 150° C., in several applications, e.g. foam drilling applications or thermally oil recovery applications. Furthermore, it is usually important in several applications, that the surfactant and the produced foam exhibit sufficient stability against higher salt concentration and contact to oily phase.

### BRIEF SUMMARY OF THE INVENTION

It is an object of the present invention to provide improved surfactants and a method for the production of foam compositions, in particular aqueous based foams, which are stable at high temperature (e.g. 150 to 250° C.). These foam compositions should exhibit a high temperature stability (e.g. for up to 100° C.) for a long period of time, particularly up to 1 day, preferably up to 10 days, often up to 60 days, and a high stability also in contact with an hydrocarbon phase. Further, requirements for the surfactants are chemical stability up to a temperature of 250° C. and a good water-solubility at room temperature, which is a precondition for several applications, e.g. pumping the solution in a well bore. Besides the resistance to high temperatures, the foaming agent should not harm the environment, should produce no residue and be easily available at low cost.

It was surprisingly found that specific sulfonates with linear alkyl chains having from 14 to 36 carbon atoms, in particular from 20 to 30, preferably from 22 to 28 carbon atoms, and having from 1 to 20 alkoxy units, preferably from 1 to 10 alkoxy units, are chemically stable and produce foams up to temperatures of 250° C. It was further found that salts (e.g. potassium chloride) enhance the foam stability. The optimal amount of salt to be used depends on the surfactant structure. Mixtures of long and short alkyl chain surfactants can also be used to adjust solubility, surface activity and foam stability.

The present invention is directed to the use of at least one linear alkylalkoxysulfonate according to formula (I)

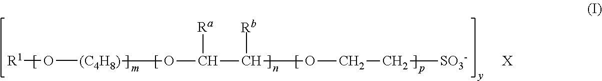

wherein:
R¹ is a linear alkyl chain having from 14 to 36 carbon atoms, preferably from 20 to 30, preferably from 22 to 30, preferably from 22 to 28, more preferably from 24 to 28;
R^{a} is, independently for each of n alkoxy units, hydrogen or methyl;
R^{b} is, independently for each of n alkoxy units, hydrogen or methyl;
with the provision that for each of the n alkoxy units at least one of R^{a} or R^{b} is not hydrogen;
m is a number from 0 to 10, preferably from 1 to 8;
n is a number from 0 to 10, preferably from 1 to 8;
p is a number from 0 to 20, preferably from 1 to 8;
with the provision that m+n+p>0;
X is selected from Na⁺, K⁺, Mg²⁺, and NH₄⁺,
y is an integer 1 or 2;
   for the production of a foam at a temperature in the range of 100° C. to 250° C., preferably in the range of 150° to 250° C., often also in the range of 180° to 250° C., preferably in the range of 200° C. to 250° C.

Furthermore, the present invention is directed to the use of a linear alkylalkoxysulfonate according to formula (I) as described above as a surfactant for the production of high temperature stable foams.

### BRIEF DESCRIPTION OF THE FIGURES

FIG. 1 shows the time t in seconds, when the first foam collapse was visible (y-axis), at temperature T in ° C. from 120 to 200° C. (x-axis) in Example 1, as described below.

### DETAILED DESCRIPTION OF THE INVENTION

A “foam” or “foam composition” according to the present invention is a composition comprising a gas, a liquid and a surfactant as lamella stabilizing agent, that is formed by trapping gas bubbles in a liquid (e.g. water), wherein the gas bubbles are separated by connected liquid films, the so called lamella. Lamellae of foam comprising a liquid, e.g. water, are normally stabilized by at least one surfactant, which shows film-forming properties. In some cases, the foam can be referred to as a colloidal suspension of a gas in a liquid. The lamellae are connected via intersections and form an interconnected network. The gas bubbles may be spherical and have weak mutual interactions—in particular in cases wherein the volume fraction of gas is less than about 74%.

In other cases with a higher gas volume fraction the shape of gas bubbles may be polyhedral deformed. In real foams, the gas bubbles are typically disordered and have a variety of bubble sizes (poly-disperse foam).

In particular, the surfactant (foaming surfactant) in terms of the present invention is a surfactant which facilitates the formation of foam, when it is dissolved, particularly in small amounts, in the liquid phase. A foaming agent or a foaming surfactant may enhance the colloidal stability of the foam by inhibition of the coalescences of bubbles.

A “high-temperature stable foam” according to the present invention means, that the foam volume is reduced by less than 10% (preferably by less than 5%, more preferably by less than 2%) after a period of 100 sec or more (preferably after a period of more than 200 sec, preferably after a period of more than 300 sec) at temperatures up to 100° C. (in particular at temperatures up to 150° C. often in the range of 150 to 250° C.). The foam stability can be described by the time before the onset of foam collapse (breaking of foam lamellae) can be observed.

Preferably, the radical R¹ in formula (I) is a linear alkyl chain having from 14 to 36 carbon atoms, preferably from 20 to 30, also preferably from 22 to 30, preferably from 22 to 28, more preferably from 24 to 28. Typically, the linear alkylalkoxysulfonate according to the invention preferably can be prepared in a manner known in principle, by alkoxylation of commercial available alcohols (e.g. fatty alcohols) or mixtures of alcohols. Thus, the number of carbon atoms of radical R¹ may refer here, in a known manner, to the average number of carbon atoms present in the alkylalkoxysulfonate, respectively refer to the maximum of distribution of the carbon atoms number in alkyl chain.

The linear alkylalkoxysulfonates used according to the present invention normally exhibit a block structure with the general structure given in formula (I). Typically, the linear alkylalkoxysulfonates used according to the present invention comprise m butoxy groups of the general formula —O—(C₄H₈); n propoxy groups of the general formula —O—CH₂—CH(CH₃)—; and p ethoxy groups of the general formula —O—CH₂CH₂—.

The formula of the propoxy group —O—CH₂—CH(CH₃)— here is expressly intended to include units also of the formula —O—CH(CH₃)—CH₂)—, thus the inverse orientation of the alkoxy group in the linear alkylalkoxysulfonates is included. Typically, both orientations may be represented in a surfactant molecule. Depending on the reaction conditions (e.g. basic or acid catalysis), the alkoxy units (e.g. propoxy units) normally are incorporated predominantly into one of the above described orientations. Furthermore, the butoxy group can be linear or branched after the polymerisation of e.g. butylenoxid. Preferably the butoxy group is branched.

Linear alkylalkoxysulfonates used according to the present invention can be prepared in a manner in principle known by a skilled person. Normally the alcohol R₁-OH is reacted with alkylene oxide (e.g. ethylene oxide, propylene oxide, butylene oxide) using an alkoxylation catalyst. The synthesis can also be started from a mixture of alkyl alcohols with a distribution of alkyl chain length, wherein normally the chain length of the maximum of distribution is mentioned (e.g. C_{24/26} alkyl alcohol or C_{16/18} alkyl alcohol). The way by which alkoxylation reactions are carried out is known to the skilled person. It is likewise known to the skilled person that the molecular weight distribution of the alkoxylates can be influenced by the reaction conditions, in particular the choice of catalyst. Afterwards the alkoxylated alcohol can e.g. be reacted with thionylchloride (e.g. in chlorobenzene) giving the chloride product. After that a reaction with e.g. sodium sulfite can follow, which gives the desired sulfonate.

The numbers m, n, p refer to the average value of the alkoxy (e.g. butoxy, propoxy, ethoxy groups) present in the alkylalkoxysulfonate, where the average value does not have to be a natural number, but may also be any desired rational number.

In particular the total number of alkoxy units (m+n+p) is a number in the range of 1 to 20, preferably 1 to 10. In a preferred embodiment m=0, n=0, and p is a number from 1 to 20, preferably from 1 to 10, preferably from 1 to 8. In a further preferred embodiment m=0, n is a number from 1 to 10, preferably from 1 to 8, and p is a number from 1 to 10, preferably from 1 to 8. In a further preferred embodiment m is a number von 1 to 8, n is a number from 1 to 8, and n is a number from 1 to 8.

In a further embodiment of the invention a linear alkylalkoxysulfonate according to formula (I) is used comprising more ethoxy units than propoxy units, wherein in particular the ratio of propoxy units and ethoxy units (n/p) is in the range of 0 to 1.

In one embodiment of the invention at least one linear alkylalkoxysulfonate according to formula (I) is used, wherein R¹ is a linear alkyl having from 20 to 30 carbon atoms, preferably from 22 to 28, and wherein m+n+p is a number in the range of 1 to 20, preferably 1 to 10.

In one embodiment, at least one linear alkylalkoxysulfonate according to formula (I) as described above can be used, wherein R¹ is a linear alkyl chain having from 20 to 30 carbon atoms, preferably from 22 to 28, m=0, n=0 and p is a number from 2 to 8.

In one embodiment, at least one linear alkylalkoxysulfonate according to formula (I) as described above can be used, wherein R¹ is a linear alkyl chain having from having from 20 to 30 carbon atoms, preferably from 22 to 28, m=0, n is a number from 2 to 8, and p is a number from 2 to 8.

Preferably, the present invention is directed to the use of an aqueous composition comprising at least one linear alkylalkoxysulfonate according to formula (I) as described above for the production of foams as described above.

In an embodiment the invention is directed to the use of an aqueous composition comprising at least one linear alkylalkoxysulfonate according to formula (I) for production of foam at a temperature in the range of 100° C. to 250° C., preferably in the range of 150° to 250° C., preferably in the range of 180° C. to 250° C., more preferably in the range of 200° C. to 250° C. and a pressure in the range of 1 to 100 bar.

Particularly, the aqueous composition used according to the present invention may comprise (or consist of):
10-99.99% (w/w), preferably 9099.99% (w/w) water,
0.01-10% (w/w), preferably 0.01 to 5% (w/w), more preferably 0.01 to 1 (w/w), more preferably 0.05 to 0.5% (w/w) of at least one linear alkylalkoxysulfonate according to formula (I) as described above,
0-25% (w/w) of at least one further additive.

The composition often consists of 90 to 99% (w/w) of water, 0.01 to 10% (w/w) of at least one alkylalkoxysulfonate. To this composition further additives (up to 25%) can be added.

Unless otherwise defined, all amounts given % (w/w) are related to the overall aqueous composition.

In an embodiment the aqueous composition described above comprises 0.01-10% (w/w), preferably 0.01 to 5% (w/w), more preferably 0.01 to 1% (w/w), more preferably 0.05 to 0.5% (w/w) of at least one linear alkylalkoxysulfonate according to formula (I) as described above.

In one embodiment the aqueous composition described above may comprise a mixture of at least two linear alkylalkoxysulfonates according to formula (I) as described above.

The further additives of aqueous composition used according to the present invention may be for example selected from:
i) water-soluble inorganic salts (for example sodium chloride, potassium chloride, magnesium chloride);
ii) co-solvents selected from alcohols, ethoxylated alcohols, e.g. butyl diglycol, ethers and esters;
iii) additional surfactants (co-surfactants);
iv) thickeners, e.g. layered silica.

Particularly, the amount of further additives is in the range of 0.01 to 25% (w/w), preferably of 0.01 to 10% (w/w), preferably of 0.1 to 5% (w/w).

In an embodiment of the invention the aqueous composition described above comprises as further additive at least one water-soluble inorganic salt in an amount of 0.01 to 25% (w/w), preferably 0.01 to 10% (w/w). In particular the water-soluble inorganic salt is selected from sodium chloride, potassium chloride and magnesium chloride. A “water-soluble compound” in terms of the present invention means a compound, which exhibits solubility in water at normal temperature (e.g. 25° C.) of more than 10 g/l.

In particular the aqueous composition described above can further comprise a cosolvent, which may be selected from a polar water-miscible solvent. In particular the cosolvent may be selected from alcohols, preferably methanol, ethanol, isopropanol, butanol, butyl monoglycol, butyl diglycol, butyl triglycol, ethers, preferably dimethyl ether, diethyl ether, dipropyl ether, methylethyl ether, methylpropyl ether, ethylpropyl ether, glycol ethers and esters, preferably ethyl acetate, n-butyl acetate, propylene based glycol esters. Suitable cosolvents may also be mixtures or combinations of the solvents mentioned above.

In an embodiment of the invention the aqueous composition described above comprises as further additive 0.01 to 25% (w/w), preferably 0.01 to 10% (w/w), preferably from 0.1 to 5% (w/w) of at least one co-solvent selected from alcohol, ether and ester.

The aqueous composition described above can further comprise as further additive a surfactant (co-surfactant) additional to the linear alky alkoxy sulfonate described in formula (I), in particular this co-surfactant exhibits more hydrophilic properties in comparison to the linear alkylalkoxysulfonate. In particular the co-surfactant may be at least one surfactant selected from the group consisting of alkyl sulfonates (e.g. cumolsulfonate, dodecylsulfonate), alkyl sulfates (e.g. dodecyl sulfate, myristyl sulfate, palmityl sulfate, stearyl sulfate), alkylaryl sulfonates, alkylaryl sulfates, alkylalkoxysulfonates, alkylalkoxy sulfates, alkylarylalkoxy sulfonates, alkylarylalkoxy sulfates, alcohol ethoxylates, alkyl phosphates, alkylalkoxy phosphates, alkyl polyglucosides and sorbitan fatty acid esters. Besides more hydrophilic co-surfactants in comparison to the linear alkylalkoxysulfonates, less hydrophilic ones can be part of the aqueous composition like saturated or unsaturated C₅₋₂₀-alcohols (e.g. pentanol, hexanol, decanol, dodecanol, oleylalcohol, C_{16/18}-alcohol), alkylamines with alkyl chain lengths from C₈ to C₁₈, fatty acids (e.g. oleic acid and stearic acid), and salts of fatty acids (e.g. sodium salt of stearic acid). Particularly, the co-surfactants mentioned above include a C₈₋₂₀-alkyl chain. The above mentioned surfactants can preferably be added in form of their salts, e.g. their alkali metal or earth alkali metal salts.

In a preferred embodiment, the aqueous composition used according to the present invention comprise as further additive a least one co-surfactant selected from the group C₈₋₂₀-alkyl sulfonates, C₈₋₂₀-alkyl sulfates, C₈₋₂₀-alkylaryl sulfonates, C₈₋₂₀-alkylaryl sulfates, C₈₋₂₀-alkylalkoxy sulfonates, C₈₋₂₀-alkylalkoxy sulfates, C₈₋₂₀-alkylarylalkoxy sulfonates, C₈₋₂₀-alkylarylalkoxy sulfates more preferably at least one C₈₋₂₀-alkylaryl sulfonate or C₈₋₂₀-alkylarylalkoxy sulfonate.

Particularly, the aqueous composition used according to the present invention comprises at least one co-surfactant in an amount of 0.01 to 10% (w/w), preferably 0.01 to 1% (w/w), preferably from 0.1 to 0.9% (w/w).

In another embodiment the aqueous composition comprises said alkylalkoxysulfonate(s) according to formula (I) as sole surfactant.

In a preferred embodiment the invention is directed to the use as described above, wherein the foam is used for washing and cleaning, formation of micro-emulsions, metal production or oil recovery applications.

Furthermore, the present invention is directed to a method of producing a foam, in particular a high-temperature stable foam, comprising the following steps
a) Preparation of an aqueous composition by adding (e.g. dissolving) at least one linear alkylalkoxysulfonate according to formula (I) in water

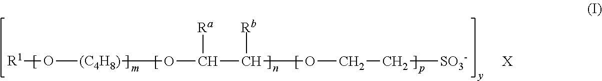

wherein:
R¹ is a linear alkyl chain having from 14 to 36 carbon atoms, preferably from 20 to 30, preferably from 22 to 30, preferably from 22 to 28, more preferably from 24 to 28;
R^{a} is, independently for each of n alkoxy units, hydrogen or methyl;
R^{b} is, independently for each of n alkoxy units, hydrogen or methyl;
with the provision that for each of the n alkoxy units at least one of R^{a} or R^{b} is not hydrogen;
m is a number from 0 to 10, preferably from 0 to 8, preferably from 1 to 8;
n is a number from 0 to 10, preferably from 1 to 8;
p is a number from 0 to 20, preferably from 1 to 8;
with the provision that m+n+p>0;
X is selected from Na⁺, K⁺, Mg²⁺, and NH₄,
y is an integer 1 or 2;
b) Optionally addition of at least one further additive to the aqueous composition;
c) Contacting the aqueous composition (obtained in step a or if applicable in step b) with a gas;
   wherein the method is carried out at a temperature in the range of 100° C. to 250° C., preferably in the range of 150° to 250° C., often in the range of 180° C. to 250° C., more preferred in the range of 200° C. to 250° C.

Typically at least one step, preferably step c, of the method described above is carried out at a temperature in the range of 100° C. to 250° C., preferably in the range of 150° to 250° C., often in the range of 180° C. to 250° C., more preferred in the range of 200° C. to 250° C.

In another embodiment step c) can be carried out at a temperature in the range of 15° C. to 30° C. and afterwards the aqueous composition (respectively the foam) is brought to a temperature in the range of 100° C. to 250° C., preferably in the range of 150° to 250° C., often in the range of 180° C. to 250° C., more preferred in the range of 200° C. to 250° C.

In a preferred embodiment of the invention contacting the aqueous composition (obtained in step a) or if applicable in step b)) with a gas (step c) is carried out by injection of a gas into the aqueous composition.

The method described above, preferably step c, can in particular be carried out at a pressure in the range of 1 to 100 bar.

Preferably, the method of producing foams is carried out at a temperature in the range of 100° C. to 250° C., preferably in the range of 150° to 250° C., often in the range of 180° C. to 250° C. More preferably contacting the aqueous composition with a gas (step c) is carried out at a temperature in range of 100° C. to 250° C., preferably in the range of 150° to 250° C., often in the range of 180° C. to 250° C., more preferably in the range of 200° C. to 250° C.

The further additive may be selected from water-soluble inorganic salts and cosolvents, co-surfactants, and thickener. In view of the further additive used in the inventive method the preferred embodiments mentioned before in connection with the use of linear alkyalkoxysulfonates for the production of foam can also be applied.

The gas used in step c is preferably selected from nitrogen, carbon dioxide, steam, water vapour, natural gas, methane, ethane, propane, fuel gas, air and mixtures thereof, preferably selected from air, steam, carbon dioxide, nitrogen and mixtures thereof.

More preferably the gas is selected from air, steam, carbon dioxide, nitrogen and mixtures thereof.

In particular at least one linear alkylalkoxysulfonate as described above in connection with the use for production of foam can be preferably used in the inventive method of producing foams.

Particularly, preferred embodiments of the aqueous composition described above in view of use of linear alkylalkoxysulfonate for production of foam can be used in the inventive method of producing foams.

Preferably the present invention is directed to a method of producing a foam as described above, wherein 0.01 to 10% (w/w) of at least one co-surfactant is added as further additive to the aqueous composition.

In an embodiment the present invention is directed to a method of producing a foam comprising the following steps:
a) Preparation of an aqueous composition by adding (e.g. dissolving) 0.01 to 10% (w/w) of at least one linear alkylalkoxysulfonate according to formula I in water

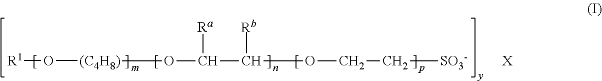

wherein:
R¹ is a linear alkyl chain having from 20 to 30, preferably from 22 to 30, preferably from 22 to 28, more preferably from 24 to 28;
R^{a} is, independently for each of n alkoxy units, hydrogen or methyl;
R^{b} is, independently for each of n alkoxy units, hydrogen or methyl;
with the provision that for each of the n alkoxy units at least one of R^{a} or R^{b} is not hydrogen;
m is a number from 0 to 10, preferably from 0 to 8, preferably from 1 to 8;
n is a number from 0 to 10, preferably from 1 to 8;
p is a number from 0 to 20, preferably from 1 to 8;
with the provision that m+n+p>0;
X is selected from Na⁺, K⁺, Mg²⁺, and NH₄⁺,
y is an integer 1 or 2;
b) Addition of at least one co-surfactant in an amount of 0.01 to 10% (w/w), preferably 0.01 to 1% (w/w), preferably from 0.1 to 0.9% (w/w) to the aqueous composition;
c) Injection of a gas into the aqueous composition (obtained in step a and if applicable in step b), at a temperature in the range of 100° C. to 250° C., preferably in the range of 150° to 250° C., preferably in the range of 180° C. to 250° C., more preferably in the range of 200° C. to 250° C., and preferably under a pressure in the range of 1 to 100 bar.

In a further aspect the present invention is directed to a foamable, aqueous composition comprising
10 to 99.99% (w/w), preferably 90 to 99.9% (w/w) water,
0.01 to 10% (w/w) of at least one linear alkylalkoxysulfonate according to formula (I),

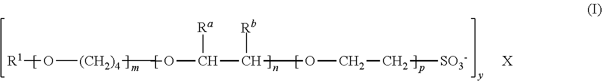

wherein:
R¹ is a linear alkyl chain having from 20 to 36 carbon atoms, preferably from 22 to 30, preferably from 24 to 30, preferably from 25 to 30, preferably from 25 to 28;
R^{a} is, independently for each of n alkoxy units, hydrogen or methyl;
R^{b} is, independently for each of n alkoxy units, hydrogen or methyl;
with the provision that for each of the n alkoxy units at least one of R^{a} or R^{b} is not hydrogen;
m is a number from 0 to 10; preferably from 0 to 8, preferably from 1 to 8;
n is a number from 0 to 10; preferably from 1 to 8;
p is a number from 0 to 20; preferably from 1 to 8;
with the provision that m+n+p>0;
X is selected from Na⁺, K⁺, Mg²+, and NH₄⁺,
y is an integer 1 or 2;
0 to 25% (w/w) of at least one further additive.

The composition often consists of 90 to 99% (w/w) of water, 0.01 to 10% (w/w) of at least one alkylalkoxysulfonate. To this composition further additives (up to 25%) can be added.

Unless otherwise defined, all amounts given % (w/w) are related to the overall aqueous composition.

Preferably, the foamable aqueous composition described above comprises 0.01 to 10% (w/w), preferably 0.01 to 5% (w/w), more preferably 0.01 to 1% (w/w), more preferably 0.05 to 0.5% (w/w) of at least one linear alkylalkoxysulfonate according to formula (I).

Furthermore, the preferred embodiments described before in connection with use of the aqueous composition for production of foam at high temperature can also be applied for the above described inventive foamable aqueous composition.

The present invention is illustrated in further detail by the following examples.

### EXAMPLE 1

### Foam Formation up to 200° C.

An autoclave with windows on the front and on the backside was used to observe foam-ability, foam structure and foam stability as function of temperature and time by a video camera. The temperature was adjusted by electrical heating units. Pictures and short videos were taken for documentation. Nitrogen was used to apply a maximum pressure of 80 bar for keeping the solution liquid. Foam was created by nitrogen injection into the aqueous surfactant solution (50 ml), which was stirred mechanically (1300 rpm). The concentration of the surfactant was 1 g/l. A frit with 2 μm pore size was placed at the end of the injection tube to create small air bubbles. After nitrogen injection stirring was stopped and foam stability was detected.

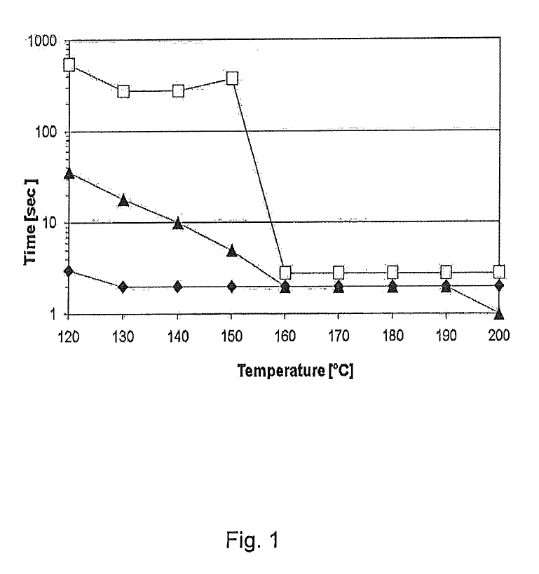

At different temperatures up to 200° C. the time was notified when the first indication of foam collapse was visible (see y-axis of FIG. 1).

FIG. 1 shows the time t in sec, when the first foam collapse was visible (y-axis), at temperature T in ° C. from 120 to 200° C. (x-axis), wherein:
the open squares (□) indicate the C16/C18-6PO-2EO-SO₃Na,
the close triangles (568) indicate the C12-Phenol-6PO-2EO-SO₃Na and
the close lozenges (♦) indicate the iC17-6PO-2EO-SO₃Na surfactant.

EO stands for ethoxy unit(s), PO stands for propoxy unit(s) and BO stand for butoxy unit(s). C with number describes the alkyl chain of surfactant, e.g. C16 means alkyl chain with 16 carbon atoms, iC17 means iso-alkyl chain with 17 carbon atoms.

As can be seen from the results in FIG. 1, in comparison to different surfactant structures with nearly the same number of C-atoms in the hydrophobic molecular part, linear alkyl chains perform very well. Branching was expected to lower the surfactant packing density at the air/water interface which destabilizes the foam lamellae.

It can bee seen in FIG. 1, that the foam comprising C16/C18-6PO-2EO-SO₃Na is stable for about 400 sec at 150° C., but the foams comprising the comparative surfactants C12-Phenol-6PO-2EO-SO₃Na and iC17-6PO-2EO-SO₃Na collapse at a temperature of 150° C. after 2 and 5 sec. At a temperature of 120° C. the foam comprising C16/C18-6PO-2EO-SO₃Na collapses after 500 sec, the foam comprising C12-Phenol-6PO-2EO-SO₃Na collapses after 35 sec and the foam comprising iC17-6PO-2EOSO₃Na collapses after 3 sec.

### EXAMPLE 2

### Foam Formation at 250° C.

At a temperature of 250° C. the sodium salts of linear C24/26-polyethoxy-sulfonates with different numbers of ethoxy units (EO) and at different concentrations of potassium chloride (KCl) were compared by their foaming behaviour. The foams were created in the same way as described in example 1. The foam stability (time before the foam starts to collapse) was determined as described in example 1. The concentration of the surfactant was 1 g/l. The results are shown in Table 1.

TABLE 1
Start of foam collapse at 250° C.

| | KCl [g/l] | | | | |
|---|---|---|---|---|---|
| | 0 | 5 | 10 | 20 | 50 |
| | Start of foam collapse [sec] | | | | |
| C24/26 0EO | 0 | 0 | 0 | 0 | 0 |
| C24/26 2EO | 20 | 110 | 184 | 216 | 108 |
| C24/26 4EO | 40 | 110 | 200 | 130 | 90 |
| C24/26 6EO | 134 | 125 | 224 | 202 | — |
| C24/26 8EO | 224 | 260 | 54 | — | — |

As can be seen from the results in Table 1, the foam stability depends on the number of ethoxy units (EO number). The addition of salt may shield the charge of the anionic sulfonate groups which results in a tighter packing at the air/water interface. lit can be predicted that foam stability increases in presence of salt.

In case of C22-2EO-SO₃Na, it was found that foam stability continuously increases with rising amount of KCl and reaches its highest value at about 100 g/l. However, in some cases, e.g. C22-5EO-SO₃Na or C24/26-4EO-SO₃Na it was found that the foam stability goes through a maximum with increasing amounts of salt. This effect may be related to salting out effects depending on EO units and the alkyl chain length as well as potentially complexing phenomena in case of C22-5 EO-SO₃Na.

### EXAMPLE 3

### Influence of Alkyl Chain Length on Foam Stability

The foam stability of foams comprising alkylalkoxy-sulfonates with different alkyl chain length was determined at temperatures in the range of 200 to 250° C. as described in Example 2, wherein the concentration of the surfactant was 1 g/l. The results were summarized in Table 2.

TABLE 2
Start of foam collapse at 200° C. and 250° C.

Temperature [° C.]

| | |
|---|---|
| 200 | 250 |

| | |
|---|---|
| | Start of foam collapse |
| Surfactant | [sec] |

| | | |
|---|---|---|
| C_{16/18}-4PO-2EO-S0₃Na | 135 | 2 |
| C₂₂-2EO-S0₃Na | 300 | 3 |
| C_{24/26}-2EO-S0₃Na | 300 | 25 |

It was found that foam stability increases with increasing alkyl chain length of the surfactant.

### EXAMPLE 4

### Foam Stability Using Surfactant Mixtures

The foam stability of C_{24/26}-4EO-SO₃Na in combination with an alkyl-aryl-alkoxy-sulfonate (C₁₂-Phenol-6PO-2EO-SO₃Na) in different ratios was determined at temperatures in the range of 200 to 250° C. as described in Example 2, wherein the concentration of the surfactant was 1 g/l. The weight ratio of surfactant was 9:1 and 8:2. The results were summarized in Table 3.

TABLE 3
Start of foam collapse at 200° C. and 250° C.

| | Temperature | |
|---|---|---|
| | [° C.] | |
| | 200 | 250 |
| | Start of foam | |
| | collapse | |
| Surfactant | [sec] | |
| C_{24/26}-4EO-S0₃Na | 550 | 40 |
| C_{24/26}-4EO-S0₃Na + C₁₂-Phenol-6PO-2EO-SO₃Na (9:1) | 1600 | 110 |
| C_{24/26}-4EO-S0₃Na + C₁₂-Phenol-6PO-2EO-SO₃Na (8:2) | 1000 | 40 |

The results show that foam stabilizing effects can also be achieved by using surfactant mixtures including an alkylalkoxysulfonate and at least one further surfactant with a shorter hydrophobic chain.

### EXAMPLE 5

### Influence of Additives

The foam stability of C22-2EO-SO₃Na surfactant in combination with layered silica clay mineral and salt was determined at temperatures in the range of 200 to 250° C. as described in Example 2, wherein the concentration of the surfactant was 1 g/l. The compositions and the results concerning foam stability at T=200° C. and T=250° C. were summarized in Table 4. Laponite RD was used as layered silica, potassium chloride (KCl) was used as salt.

TABLE 4
Start of foam collapse at 200° C. and 250° C.

| | Temperature [° C.] | |
|---|---|---|
| | 200 | 250 |
| | Start of foam | |
| surfactant + additive | collapse [sec] | |
| C₂₂-2EO-S0₃Na (without additive) | 300 | 3 |
| C₂₂-2EO-S0₃Na + 5 g/l KCl | 820 | 5 |
| C₂₂-2EO-S0₃Na + 50 g/l KCl | 1800 | 230 |
| C₂₂-2EO-S0₃Na + 0.5 g/l Laponite RD | 1700 | 37 |
| C₂₂-2EO-S0₃Na + 0.5 g/l Laponite RD + 50 g/l KCl | 2050 | 230 |

The clay mineral can thicken the water phase between the foam lamella in order to reduce water drainage and to enhance foam stability. Clay mineral (Laponite RD) exhibits a foam stabilizing effect.

### EXAMPLE 6

### Chemical Stability

Storage of an aqueous solution of 1 g/l C22-2EO-SO₃Na for a period of 12 hours at 250° C. and a pressure of 62 bar had no influence on foaming behaviour.

1.-14. (canceled)

15. A method for the production of a foam at a temperature in the range of 100° C. to 250° C. using at least one linear alkylalkoxysulfonate according to formula (I) 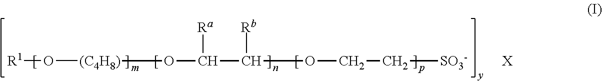
wherein:
R¹ is a linear alkyl chain having from 14 to 36 carbon atoms;
R^{a} is, independently for each of n alkoxy units, hydrogen or methyl;
R^{b} is, independently for each of n alkoxy units, hydrogen or methyl;
with the provision that for each of the n alkoxy units at least one of R^{a} or R^{b} is not hydrogen;
m is a number from 0 to 10;
n is a number from 0 to 10;
p is a number from 0 to 20;
with the provision that m+n+p>0;
X is selected from Na⁺, K⁺, Mg²⁺, and NH₄⁺,
y is an integer 1 or 2.

16. The method of claim 15, wherein R¹ is a linear alkyl chain having from 20 to 30 carbon atoms.

17. The method of claim 15, wherein an aqueous composition comprising at least one linear alkylalkoxysulfonate according to formula (I) is used for production of foams.

18. The method of claim 15, wherein an aqueous composition comprising the following components is used:
10-99.99% (w/w) water,
0.01-10% (w/w) of at least one linear alkylalkoxysulfonate according to formula (I),
0-25% (w/w) of at least one further additive.

19. The method of claim 15, wherein the aqueous composition comprises as further additive at least one water-soluble inorganic salt in an amount of 0.01 to 25% (w/w).

20. The method of claim 15, wherein the aqueous composition comprises as further additive 0.01 to 25 (w/w) of at least one co-solvent selected from the group consisting of alcohol, ether and ester.

21. The method of claim 15, wherein the aqueous composition comprising at least one linear alkylalkoxysulfonate according to formula (I) is used for production of foam at a temperature in the range of 150° C. to 250° C. and a pressure in the range of 1 to 100 bar.

22. The method of claim 15, wherein the foam is used for washing and cleaning, formation of micro emulsions, metal production or oil recovery applications.

23. A method of producing foams comprising the following steps:
a) preparing an aqueous composition by adding at least one linear alkylalkoxysulfonate according to formula (I) in water 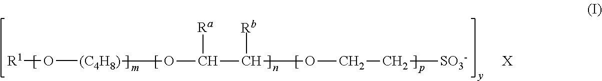
wherein:
R¹ is a linear alkyl chain having from 14 to 36 carbon atoms;
R^{a} is, independently for each of n alkoxy units, hydrogen or methyl;
R^{b} is, independently for each of n alkoxy units, hydrogen or methyl;
with the provision that for each of n alkoxy units at least one of R^{a} or R^{b} is not hydrogen;
m is a number from 0 to 1;
n is a number from 0 to 10;
p is a number from 0 to 20;
with the provision that m+n+p>0;
X is selected from Na⁺, K⁺, Mg²⁺, and NH₄⁺,
y is an integer 1 or 2;
b) optionally adding at least one further additive to the aqueous composition; and
c) contacting the aqueous composition with a gas;
wherein the method is carried out at a temperature in the range of 100° C. to 250° C.

24. The method of claim 23, wherein the contacting the aqueous composition with a gas step is carried out by injection of a gas into the aqueous composition.

25. The method of claim 23, wherein the gas is selected from the group consisting of nitrogen, carbon dioxide, steam, water vapour, natural gas, methane, ethane, propane, fuel gas, air and mixtures thereof.

26. The method of claim 23, wherein 0.01 to 10% (w/w) of at least one co-surfactant is added as further additive to the aqueous composition.

27. The method of claim 23, wherein the contacting the aqueous composition with a gas step is carried out at a temperature in the range of 150° C. to 250° C.

28. A foamable aqueous composition comprising:
10 to 99.99% (w/w) water,
0.01 to 10% (w/w) of at least one linear alkylalkoxysulfonate according to formula (I), 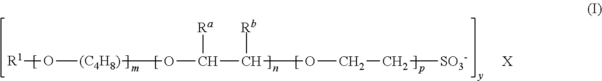
wherein:
R¹ is a linear alkyl chain having from 20 to 36 carbon atoms;
R^{a} is, independently for each of n alkoxy units, is hydrogen or methyl;
R^{b} is, independently for each of n alkoxy units, is hydrogen or methyl;
with the provision that for each of n alkoxy units at least one of R^{a} or R^{b} is not hydrogen;
m is a number from 0 to 10;
n is a number from 0 to 10;
p is a number from 0 to 20;
with the provision that m+n+p>0;
X is selected from Na⁺, K⁺, Mg²⁺, and NH₄⁺,
y is an integer 1 or 2; and
0 to 25 (w/w) of one or more further additives.
